(19) United States
(12) Patent Application Publication
(10) Pub. No.: US 2025/0223308 A1
(43) Pub. Date: Jul. 10, 2025

(54) METAL COMPLEX AND FORMULATION FOR THE PREPARATION OF METAL OXIDE OPTICAL LAYERS

(71) Applicant: MERCK PATENT GMBH, DARMSTADT (DE)

(72) Inventors: OLIVER DOLL, DIETZENBACH (DE); HAGAI ARBELL, MEVASERET ZION (IL); HENNING SEIM, DARMSTADT (DE); OLEG CHASHCHIKHIN, PETAH TIKVA (IL); NINA LIDICH, JERUSALEM (IL)

(21) Appl. No.: 19/093,764

(22) Filed: Mar. 28, 2025

Related U.S. Application Data
(63) Continuation of application No. PCT/EP2023/076448, filed on Sep. 26, 2023.

(30) Foreign Application Priority Data
Sep. 29, 2022 (EP) 22198795.1

(51) Int. Cl. C07F 15/00; C09D 7/63; C09D 163/04

(52) U.S. Cl. CPC C07F 15/00; C09D 7/63; C09D 163/04

(57) ABSTRACT

A metal complex, a formulation comprising metal complex and a method for preparing a metal oxide optical layer using said formulation and metal complex. The obtained metal oxide optical layers are particularly suitable for applications in optical devices such as, for example, in augmented reality (AR) and/or virtual reality (VR) devices.

### CROSS-REFERENCE TO RELATED APPLICATIONS

This application is a Continuation under 35 USC § 111(a) of International Patent Application No. PCT/EP2023/076448 filed Sep. 26, 2023, which claims priority to EP Application No. 22198795.1 filed Sep. 29, 2022. The entire contents of these applications are incorporated herein by reference in their entirety.

### FIELD OF THE INVENTION

The present invention relates to a metal complex, a formulation and a method for preparing metal oxide optical layers. Said metal complex, formulation and method according to the present invention are particularly suitable for the preparation of metal oxide optical layers for optical applications or devices such as, for example, diffractive gratings for augmented reality (AR) and/or virtual reality (VR) devices. The metal oxide optical layers show (a) favorable optical properties such as high refractive index (RI) of >1.7, preferably >2.0, at wavelengths of <520 nm and low absorption (optical loss); (b) favorable mechanical properties such as low shrinkage; (c) favorable coating properties such as dense layer and flat surface structure; and/or (d) favorable filling properties such as homogeneous filling of topographical features on patterned substrates.

The embodiments of the present invention allow the preparation of metal oxide optical layers on the surface of both patterned and non-patterned substrates. The metal oxide optical layers contain a metal oxide embedded in a cured matrix. The layers may form various structures such as, for example, layers covering a surface of a non-patterned substrate and/or fillings covering topographical features such as, for example, gaps on the surface of a patterned substrate, thereby providing highly refractive optical structures. In particular, the embodiments of the present invention allow the preparation of advanced optical gap filling with low overburden, thereby enabling an easy and cost efficient mass production of complex optical devices by avoiding typical problems occurring when layer deposition or gap filling is performed by physical vapor deposition (PVD) or chemical vapor deposition (CVD) techniques such as, for example, incomplete or excessive gap filling due to unfavourable deposition and layer growth characteristics such as, for example, decreased or increased deposition or growth rates at corners and edges.

The embodiments of the present invention are particularly suitable for the preparation of metal oxide optical layers having high refractive index for optical devices such as, for example, for diffractive gratings in AR and/or VR devices.

Finally, the present invention provides an optical device, preferably an AR and/or VR device, comprising a metal oxide optical layer, which is obtainable by the method according to the present invention.

### BACKGROUND OF THE INVENTION

Leading edge optical devices typically include optical gratings made from composite materials having a substrate as a support and complex and interlaced patterns thereon, the patterns being made up of different layers or stacks of layers. Usually, the creation of such complex and interlaced patterns demands for structuring processes, which become increasingly challenging with decreasing size of structural dimensions to be prepared.

In addition to a wide range of possible uses in various fields of application, such as in spectrometers or in optical storage systems (CD, DVD, etc.), diffractive gratings are the core components of so-called XR devices, mostly glasses. In this context, R stands for the term reality and X denotes different attributes such as, for example, virtual, augmented, mixed and so forth. Hence, diffractive gratings form part of the core of the so-called optical engine in XR devices, specifically in augmented reality and mixed reality glasses. Virtual reality glasses, when built as a head mounted display, are often composed of a conventional liquid crystal (LC) organic light emitting diode (OLED) display being embedded in the device, and thus do not necessarily require diffractive gratings. In contrast, augmented and mixed reality glasses are designed that way to enable consumers to obtain visual impressions of their environment, at its best as if they would not wear any glasses at all. However, they also make it possible to provide and serve digital information and to also project it into the field of vision of individuals. Additional digital information is gathered from recognizing and analyzing the environment, the individual inspects or takes a look currently at. In order to convey and project supporting digital information into the eyes of an individual, the augmented or mixed reality glasses are equipped with an information supply unit, which is coupled to an optical waveguide system that transports the optically coded supporting information through it directly to the lens of the glasses. Here, the information passes a diffractive grating, which couples the incident light into the lens and splits it according to its angular information and its spectral bands by diffraction. After incoupling of the light, the lens serves as waveguide enabling transport of the light to and into the pupil of an individual. The location of light incoupling is independent of any preferred position and thus of the implication of technical needs. The direction of traversal of light within the lenses is determined by the diffractive grating diffracting or splitting the light. At certain positions in the lens, a second and a third diffractive grating serves for changing the direction of light traversal and thereby enforcing the light to be projected into pupil of the user. The light traversal in the glasses is accomplished by total internal reflection (TIR) of the light, thus bouncing several times between the glass interfaces until reaching another diffractive grating, which changes the internal TIR direction of the light (see FIG. 2). The second and third grating are geometrically aligned in different directions with respect to the first and incoupling grating, e. g. by a certain angular distortion of the longitudinal axis, thus allowing to change the direction of propagation of totally internally reflected light. Needless to say, the lens itself or the material of which lenses are made of shall not be absorbing. Otherwise, the supportive information never reaches the pupil of the user or only with strongly depleted light intensity. The process works regardless of the use of reflection or transmission gratings. Usually, the lenses are equipped with both types of gratings to properly guide the light. It should also be mentioned that there are differences in the optical performance of reflection and transmission gratings, which, however, are of no further interest in the context of the current invention. The basic structure of the gratings is very similar, which is more important at this point.

Nevertheless, there are different designs and structures such as surface relief (SR) or volume phase holographic (VPH) gratings to achieve waveguide. Both types are very similar in appearance. In the simplest case, the gratings are somehow mounted onto the surface of a waveguiding material, here the lens. The grating itself is composed of an array of fine structures, mostly trenches of a first material type Material 01 with a refractive index RI 01, however, not limited thereto. The geometrical shape of the trenches may be manifold, from rectangular, over V-shaped trenches, U-shaped and there like. The width, including structures with different widths, the geometrical form of the trenches, their pitch as well as their depth, including different depths, are specially designed to influence the diffraction pattern of the incident light to be diffracted.

In case of SR gratings (SRGs), the trenches or structures of a first material type (Material 01) having a refractive index (RI 01) are filled by a second material type (Material 02) having a refractive index (RI 02), wherein RI 02 is incrementally different from RI 01 (see FIGS. 1 and 3). For the sake of completeness, it should be mentioned that Material 01 or Material 02 may be composed of a stack of structured layers, each containing a different material composition with different refractive index, stacked on top of each other, thereby forming Material 01 or Material 02 having an effective or graded refractive index RI 01 or RI 02, respectively. Incidentally, the (effective or graded) refractive indices RI 01 and RI 02 depend on the refractive index of the waveguide or the lens from which the glasses are made of. If a glass lens with high refractive index (n03>1.46) is used, the (effective or graded) refractive indices of Material 01 and Material 02 are considered to be higher than that of the lens itself, whereby a RI value of 2.0 can be reached and exceeded. High performance gratings, especially those of SR-type, may be manufactured using standard lithography and deposition techniques known from micro-fabrication such as, for example, the manufacturing of integrated circuits.

Such standard techniques typically include physical vapor deposition (PVD) or chemical vapor deposition (CVD) processes and often suffer from incomplete gap filling due to unfavourable deposition and/or layer growth deposition properties including increased deposition and/or growth rates at corners and edges. Such incomplete gap filling results in the formation of voids within the structures to be filled by the PVD- and CVD-materials. In addition to the formation of voids, the surface of the substrate is covered by a PVD and/or CVD layer that is almost as thick as the maximum depth of the deepest structure to be filled by the deposited gap filling material (see FIGS. 4 and 5). In some applications, however, it may be necessary to expose the surface of the substrate so that it is available for further processing. As a consequence, undesired overburden layers from PVD or CVD need to be removed, for example by chemical mechanical planarization (CMP) without harming the original substrate surface underneath. Although CMP is very well established in the process of manufacturing integrated circuits, CMP is a time consuming and costly process and can be seen as a potential economic drawback for mass production of leading-edge optical devices, particularly the mass production of diffractive gratings. It would therefore be desirable to have a solution for an advanced and cost-efficient manufacturing of optical gratings where gap filling does not require CMP (see FIG. 6).

The present invention addresses various disadvantages of the technologies for preparing optical gratings for leading edge optical devices as described above. The focus here is on improved optical properties, improved mechanical properties, improved coating properties and improved filling properties.

### OBJECT OF THE INVENTION

It is an object of the present invention to provide a metal complex, a formulation and a method for preparing metal oxide optical layers, wherein said layers are particularly suitable for optical applications and may be used in optical devices such as, for example, in diffractive gratings for AR and/or VR devices. The obtained metal oxide optical layers show (a) favorable optical properties such as high refractive index (RI) of >1.7, preferably >2.0, at wavelengths of <520 nm and low absorption (optical loss); (b) favorable mechanical properties such as low shrinkage, (c) favorable coating properties such as dense layer and flat surface structure; and/or (d) favorable filling properties such as homogeneous filling of topographical features on patterned substrates.

Moreover, it is an object of the present invention to provide a metal complex, a formulation and a method allowing an easy and cost-efficient preparation of metal oxide optical layers.

It is a further object of the present invention to enable preparation of metal oxide optical layers on the surface of both patterned and non-patterned substrates. The metal oxide layers may form various structures such as, for example, layers covering a surface of a non-patterned substrate and/or fillings covering topographical features such as, for example, gaps on the surface of a patterned substrate, thereby providing highly refractive optical structures.

Hence, it is an object of the present invention to provide a metal complex, a formulation and a method for preparing a metal oxide optical layer, which allow obtaining advanced optical gap filling with low overburden, thus enabling an easy and cost-efficient mass production of complex optical devices.

It is a further object of the present invention to provide a metal complex, a formulation and a method for preparing a metal oxide optical layer, which avoid typical problems occurring when layer deposition or gap filling is performed by PVD or CVD techniques such as, for example, incomplete or excessive gap filling due to unfavourable deposition and layer growth characteristics such as, for example, decreased or increased deposition or growth rates at corners and edges.

It is an object of the present invention that the metal complex and formulation are particularly suitable for the preparation of metal oxide optical layers having high refractive index and at the same time low absorption (optical loss) for optical devices such as, for example, for diffractive gratings in AR and/or VR devices.

Finally, it is an object of the present invention to provide an optical device, preferably an AR and/or VR device, comprising a metal oxide optical layer, which is obtainable by the method according to the present invention, and thereby shows the above-mentioned beneficial effects.

### SUMMARY OF THE INVENTION

The present inventors surprisingly found that the above objects are achieved by the following embodiments:

A metal complex comprising:
one or more, preferably two or more, metals M selected from the list consisting of V, Nb and Ta, preferably Nb; and
one or more ligands L, wherein L is an organic ligand comprising an ester group, preferably a carboxylic acid ester group, and a carboxylic acid group, which is optionally deprotonated.

A formulation comprising:
(i) a metal complex according to the present invention as metal oxide precursor;
(ii) an epoxy resin mixture; and
(iii) a photoinitiator.

A method for preparing a metal oxide optical layer comprising the following steps (a) to (c):
(a) providing a formulation according to the present invention;
(b) applying the formulation to a surface of a substrate; and
(c) converting the formulation on the surface of the substrate to a metal oxide optical layer.

Finally, an optical device is provided comprising a metal oxide optical layer, which is obtainable or obtained by the method according to the present invention, wherein the optical device is preferably an augmented reality (AR) and/or virtual reality (VR) device.

Preferred embodiments of the present invention are described hereinafter and in the dependent claims.

### BRIEF DESCRIPTION OF THE FIGURES

FIG. 1: Schematic cross-sectional view of a SR grating with a Material 01 and a Material 02, wherein the refractive index IR 01 of Material 01 is incrementally different to the refractive index IR 02 of Material 02.

FIG. 2: Schematic cross-sectional view of a SR grating enabling light diffraction (transmissive case) including propagation of diffracted light within waveguide (e.g. lens) by total internal reflection.

FIG. 3: Schematic cross-sectional view of a SR grating providing gaps (trenches) to be filled with a high refractive index material (Material 02), wherein the refractive index of Material 02 is incrementally different form the refractive index of Material 01 flanking the gaps (trenches).

FIG. 4: Schematic representation of PVD- or CVD-mediated gap filling process and removal of undesired overburden.

FIG. 5: Schematic representation of PVD- or CVD-mediated gap filling process creating and leaving voids within gaps and deposited layers.

FIG. 6: Schematic representation of gap filling process using formulations containing inventive metal complex or formulations thereof being converted to metal oxides.

FIG. 7: SEM cross-sectional view of a substrate showing surface feature filling of spin coated 5/5% (w/w) Nb citraconate/Epoxy Mixture A after prebake at 100° C. for 1 min followed by UV-curing by 365 nm light for 20 min and bake at 350° C. for 10 min as described in Example 1.

FIG. 8: SEM cross-sectional view of a substrate showing surface feature filling of spin coated Nb citraconate 5% (w/w) solution in PGME, annealed at 300° C. for 10 min as described in Example 1.

FIG. 9: SEM cross-sectional view of a substrate showing surface feature filling behavior for Nb citraconate/Epoxy Mixture A at various mixing ratios and baking temperatures as described in Example 2.

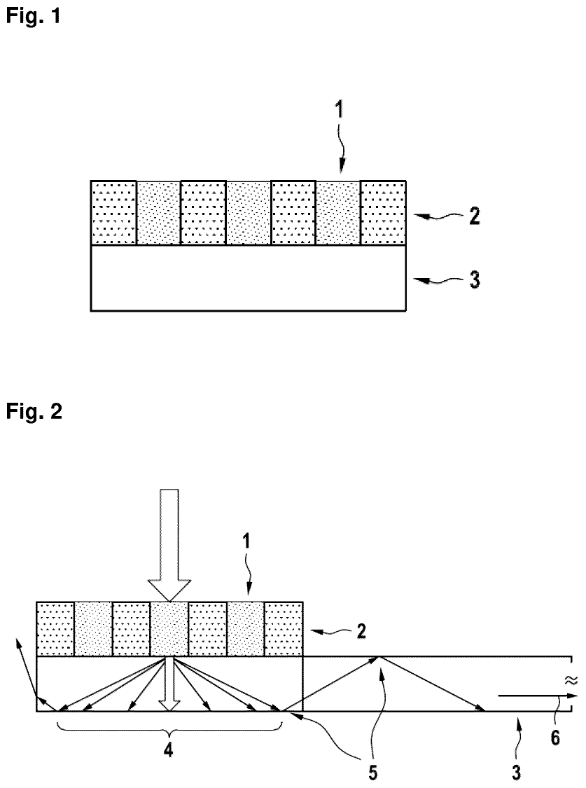

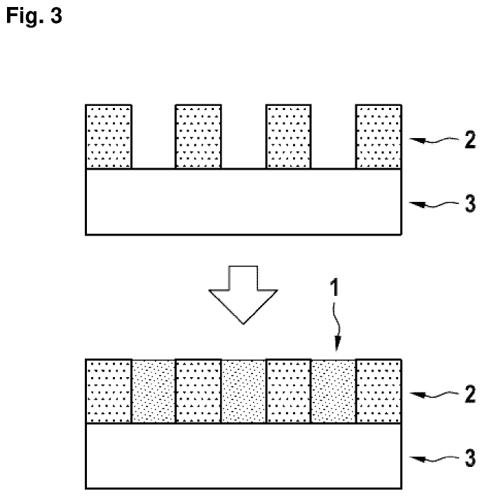

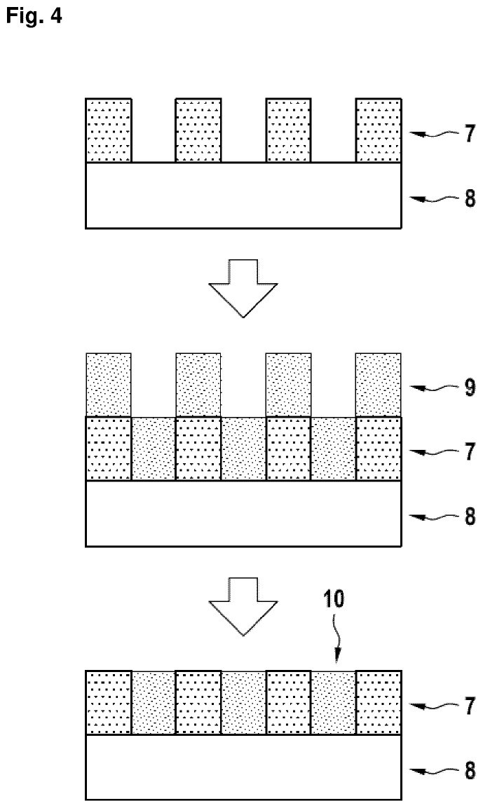

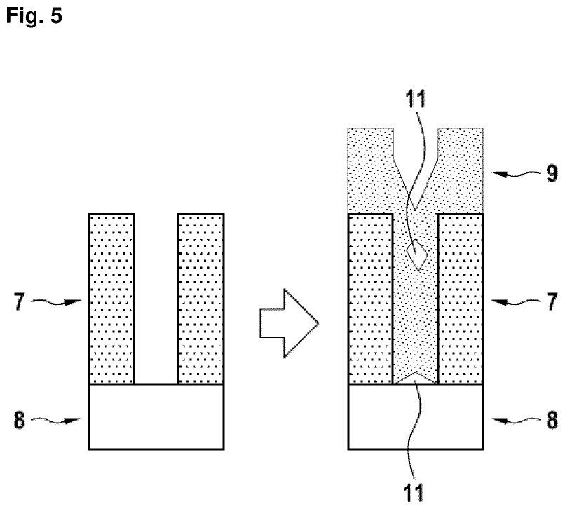

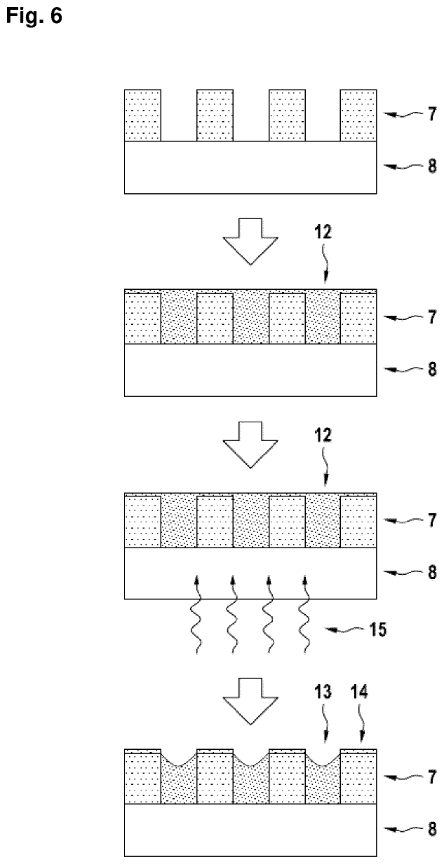

### DETAILED DESCRIPTION

### Definitions

The term “metal complex” as used herein, refers to a coordination complex consisting of one or more central metal atoms or ions, which form one or more coordination centers, and a surrounding array of bound molecules or ions as ligands, which contain one or more pairs of electrons that can be shared with the metal. In a metal complex, the metal atoms or ions typically act as Lewis acids, whereas the ligands typically act as Lewis bases. Metal complexes can be neutral, positively charged, or negatively charged. Electrically charged metal complexes may be also called complex ions.

The term “metal-organic complex” as used herein, refers to a class of metal complexes that contain metals and organic ligands, which confer solubility in organic solvents or volatility. Compounds with these properties find various applications in materials science for metal organic vapor deposition (MOCVD) or sol-gel processing. The distinct term “metal-organic complex” refers to metal-containing compounds lacking direct metal-carbon bonds, but which contain organic ligands. Metal alkoxides, metal carboxylates, metal β-diketonates, metal dialkylamides, and metal phosphine complexes are representative members of this class.

The term “ligand” as used herein, refers to ions or neutral molecules (having one or more functional groups) that bind to a central metal atom or ion to form a metal complex. The bonding with the metal generally involves formal donation of one or more of the ligand's electron pairs, often via Lewis bases. The nature of metal-ligand bonding can range from covalent to ionic. Furthermore, the metal-ligand bond order can range from one to three. Ligands are typically regarded as Lewis bases, although rare cases are known to involve Lewis acids ligands. Ligands are classified as L or X (or a combination thereof), depending on how many electrons they provide for the bond between ligand and central atom. L ligands donate two electrons from a lone electron pair, resulting in coordinate covalent bonding. X ligands donate one electron, with the central atom donating the other electron, thereby creating regular covalent bonding.

The term “alkyl” or “alkyl group” as used herein, relates to a linear, branched, cyclic or bridged cyclic alkyl group, which forms part of a structure of a chemical compound and binds via a carbon atom. An alkyl group may contain one or more heteroatoms selected from N, O, S and P. An alkyl group may be unsubstituted or substituted, preferably with one or more substituents selected from the list consisting of —C(O)R^{v}, —C(O)OR^{v}, —NR^{v}R^{w}, —OR^{v}, —R^{x}, —CN, —F and —Cl, wherein R^{v}=H, C3-C10 aryl or C1-C10 alkyl, R^{w}=H, C3-C10 aryl or C1-C10 alkyl and R^{x}=C3-C10 aryl or C1-C10 alkyl, preferably R^{v}=H, methyl, ethyl, propyl or phenyl, R^{w}=H, methyl, ethyl, propyl or phenyl and R^{x}=phenyl. An alkyl group may contain one or more functional groups, preferably selected from the list consisting of carbon-carbon double bond, carbon-carbon triple bond, amide, carbamate, carbonate, carboxylic acid, ester, ether, secondary or tertiary amine, and keto. Alkyl groups that connect two adjacent structural units in a chemical compound are referred to as “alkylene groups”.

The term “aryl” or “aryl group” as used herein, relates to a monocyclic or polycyclic aromatic group, which forms part of a structure of a chemical compound. Polycyclic aromatic groups include two or more connected aromatic ring systems, which are fixed in one plane. An aryl group may be (i) a hydrocarbon aryl group or (ii) a heteroatom containing aryl group, also referred to as heteroaryl group. Hydrocarbon aryl groups contain an aromatic ring structure made of carbon atoms, whereas heteroaryl groups contain an aromatic ring structure, which further comprises one or more heteroatoms selected from N, O, S and P. An aryl group may be unsubstituted or substituted, preferably with one or more substituents selected from the list consisting of —C(O)R^{v}, —C(O)OR^{v}, —NR^{v}R^{w}, —OR^{v}, —R^{x}, —CN, —F and —C, wherein R^{v}=H, C3-C10 aryl or C1-C10 alkyl, R^{w}=H, C3-C10 aryl or C1-C10 alkyl and R^{x}=C3-C10 aryl or C1-C10 alkyl, preferably R^{v}=H, methyl, ethyl, propyl or phenyl, R^{w}=H, methyl, ethyl, propyl or phenyl and R^{X}=methyl, ethyl, propyl or phenyl.

The term “fluorene epoxy resin” as used herein, relates to a compound having both fluorene and epoxy groups.

The term “acrylate epoxy resin” as used herein, relates to a compound having both acrylate and epoxy groups, thereby providing crosslinking functionality. An acrylate epoxy resin is a so-called dual reactive compound comprising a first reactive group (acrylate group) and a second reactive group (epoxy group).

The term “optical device” as used herein, relates to a device containing one or more optical components for forming a light beam including, but not limited to, gratings, lenses, prisms, mirrors, optical windows, filters, polarizing optics, UV and IR optics, and optical coatings. Preferred optical devices in the context of the present invention are augmented reality (AR) glasses and/or virtual reality (VR) glasses.

### PREFERRED EMBODIMENTS

### Metal Complex

The present invention relates to a metal complex, which comprises one or more, preferably two or more, metals M selected from the list consisting of V, Nb and Ta, preferably Nb; and one or more ligands L, wherein L is an organic ligand comprising an ester group, preferably a carboxylic acid ester group, and a carboxylic acid group, which is optionally deprotonated.

An ester group is derived from an acid (organic or inorganic) in which at least one —OH (hydroxy) group is replaced by an —O-alkyl (alkoxy) group.

Preferred is a metal complex, which comprises one or more metals M selected from the list consisting of V, Nb and Ta; and one or more ligands L, wherein L is an organic ligand comprising an ester group, preferably a carboxylic acid ester group, and a carboxylic acid group, which is optionally deprotonated.

More preferred is a metal complex, which comprises one or more metals M selected from the list consisting of V, Nb and Ta; and one or more ligands L, wherein L is an organic ligand comprising a carboxylic acid ester group, and a carboxylic acid group, which is optionally deprotonated.

Most preferred is a metal complex, which comprises Nb; and one or more ligands L, wherein L is an organic ligand comprising a carboxylic acid ester group, and a carboxylic acid group, which is optionally deprotonated.

Preferably, L is an organic ligand further comprising a carbon-carbon double bond.

Preferably, L coordinates via the carboxylic acid group to the one or more metals M.

Preferably, the metal complex according to the present invention is polynuclear.

In a preferred embodiment of the present invention, L is a compound represented by Formula (1), which is optionally deprotonated at the carboxylic acid group:

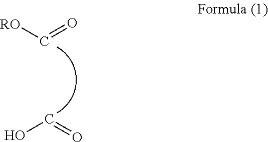

wherein:
the curved line represents a divalent hydrocarbon group having 2 to 20 carbon atoms, preferably 2 to 10 carbon atoms, more preferably 3 to 5 carbon atoms, and one or more carbon-carbon double bonds, preferably one or two carbon-carbon double bonds, more preferably one carbon-carbon double bond; and
R is an alkyl group having 1 to 20 carbon atoms, preferably 1 to 10 carbon atoms, more preferably 1 to 5 carbon atoms, or an aryl group having 5 to 20 aromatic ring atoms, preferably 5 to 10 aromatic ring atoms, more preferably 5 or 6 aromatic ring atoms, which may be substituted or unsubstituted.

Preferably, L is a compound represented by Formula (1), which is optionally deprotonated at the carboxylic acid group, wherein:
the curved line represents a divalent hydrocarbon group having 2 to 10 carbon atoms and one or two carbon-carbon double bonds; and
R is an alkyl group having 1 to 10 carbon atoms or an aryl group having 5 to 10 aromatic ring atoms, which may be substituted or unsubstituted.

More preferably, L is a compound represented by Formula (1), which is optionally deprotonated at the carboxylic acid group, wherein:
the curved line represents a divalent hydrocarbon group having 3 to 5 carbon atoms and one or more carbon-carbon double bond; and
R is an alkyl group having 1 to 5 carbon atoms or an aryl group having 5 or 6 aromatic ring atoms, which may be substituted or unsubstituted.

It is preferred in Formula (1) that substituted alkyl contains one or more substituents selected from the list consisting of —C(O)R^{v}, —C(O)OR^{v}, —NR^{v}R^{w}, —OR^{v}, —R^{x}, —CN, —F and —C, wherein R^{v}=H, C3-C10 aryl or C1-C10 alkyl, R^{w}=H, C3-C10 aryl or C1-C10 alkyl and R^{x}=C3-C10 aryl or C1-C10 alkyl, preferably R^{v}=H, methyl, ethyl, propyl or phenyl, R^{w}=H, methyl, ethyl, propyl or phenyl and R^{x}=phenyl.

It is preferred in Formula (1) that alkyl, which contains one or more functional groups, contains one or more functional groups selected from the list consisting of carbon-carbon double bond, carbon-carbon triple bond, amide, carbamate, carbonate, carboxylic acid, ester, ether, secondary or tertiary amine, and keto.

It is preferred in Formula (1) that substituted aryl contains one or more substituents selected from the list consisting of —C(O)R^{v}, —C(O)OR^{v}, —NR^{v}R^{w}, —OR^{v}, —R^{x}, —CN, —F and —Cl, wherein R^{v}=H, C3-C10 aryl or C1-C10 alkyl, R^{w}=H, C3-C10 aryl or C1-C10 alkyl and R^{x}=C3-C10 aryl or C1-C10 alkyl, preferably R^{v}=H, methyl, ethyl, propyl or phenyl, R^{w}=H, methyl, ethyl, propyl or phenyl and R^{X}=methyl, ethyl, propyl or phenyl.

In a more preferred embodiment of the present invention, L is a compound represented by Formula (2), which is optionally deprotonated at the carboxylic acid group:

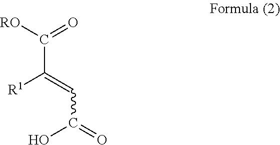

wherein:
R is an alkyl group having 1 to 20 carbon atoms, preferably 1 to 10 carbon atoms, more preferably 1 to 5 carbon atoms, or an aryl group having 5 to 20 aromatic ring atoms, preferably 5 to 10 aromatic ring atoms, more preferably 5 or 6 aromatic ring atoms, which may be substituted or unsubstituted; and
R¹ is H, an alkyl group having 1 to 10 carbon atoms, preferably 1 to 5 carbon atoms, or an aryl group having 5 to 10 aromatic ring atoms, preferably 5 or 6 aromatic ring atoms, which may be substituted or unsubstituted.

Preferably, L is a compound represented by Formula (2), which is optionally deprotonated at the carboxylic acid group, wherein:
R is an alkyl group having 1 to 10 carbon atoms or an aryl group having 5 to 10 aromatic ring atoms, which may be substituted or unsubstituted; and R¹ is H, an alkyl group having 1 to 5 carbon atoms or an aryl group having 5 or 6 aromatic ring atoms, which may be substituted or unsubstituted.

More preferably, L is a compound represented by Formula (2), which is optionally deprotonated at the carboxylic acid group, wherein:
R is an alkyl group having 1 to 5 carbon atoms or an aryl group having 5 or 6 aromatic ring atoms, which may be substituted or unsubstituted; and R¹ is H, an alkyl group having 1 to 5 carbon atoms or an aryl group having 5 or 6 aromatic ring atoms, which may be substituted or unsubstituted.

Particularly preferably, L is a compound represented by Formula (2), which is optionally deprotonated at the carboxylic acid group, wherein:
R is methyl, ethyl or propyl; and
R¹ is H, methyl or phenyl.

Most preferably, L is a compound represented by Formula (2), which is optionally deprotonated at the carboxylic acid group, wherein:
R is ethyl; and
R¹ is methyl.

It is preferred in Formula (2) that substituted alkyl contains one or more substituents selected from the list consisting of —C(O)R^{v}, —C(O)OR^{v}, —NR^{v}R^{w}, —OR^{v}, —R^{x}, —CN, —F and —C, wherein R^{v}=H, C3-C10 aryl or C1-C10 alkyl, R^{w}=H, C3-C10 aryl or C1-C10 alkyl and R^{x}=C3-C10 aryl or C1-C10 alkyl, preferably R^{v}=H, methyl, ethyl, propyl or phenyl, R^{w}=H, methyl, ethyl, propyl or phenyl and R^{x}=phenyl.

It is preferred in Formula (2) that alkyl, which contains one or more functional groups, contains one or more functional groups selected from the list consisting of carbon-carbon double bond, carbon-carbon triple bond, amide, carbamate, carbonate, carboxylic acid, ester, ether, secondary or tertiary amine, and keto.

It is preferred in Formula (2) that substituted aryl contains one or more substituents selected from the list consisting of —C(O)R^{v}, —C(O)OR^{v}, —NR^{v}R^{w}, —OR^{v}, —R^{x}, —CN, —F and —Cl, wherein R^{v}=H, C3-C10 aryl or C1-C10 alkyl, R^{w}=H, C3-C10 aryl or C1-C10 alkyl and R^{x}=C3-C10 aryl or C1-C10 alkyl, preferably R^{v}=H, methyl, ethyl, propyl or phenyl, R^{w}=H, methyl, ethyl, propyl or phenyl and R^{x}=methyl, ethyl, propyl or phenyl.

It is preferred that the metal M in the metal complex of the present invention is in an oxidation state selected from the list consisting of +I, +II, +III, +IV and +V, preferably +II, +III, +IV and +V, more preferably +V.

In a preferred embodiment of the present invention, the metal complex is represented by the following Formula (3):

MₘLₙAₐB_{b} Formula (3)
wherein:
M is at each occurrence independently from each other a metal selected from the list consisting of V, Nb and Ta, preferably Nb;
L is at each occurrence independently from each other an organic ligand comprising an ester group, preferably a carboxylic acid ester group, and a carboxylic acid group, which is optionally deprotonated;
A is a bridging ligand selected from μ-L⁻, μ-OH⁻, μ-OR⁻, μ-F⁻ μ-Cl⁻, μ-Br⁻ or μ-I⁻, wherein R is an alkyl group having 1 to 10 carbon atoms, preferably 1 to 5 carbon atoms, more preferably methyl, ethyl, propyl, butyl or pentyl; and L⁻ is a deprotonated L;
B is a bridging ligand μ-O²⁻;
m is an integer from 1 to 10, preferably 1 to 5, more preferably 1 or 2, and most preferably 1;
n is an integer from 5 to 50, preferably 5 to 25, more preferably 5 to 10, and most preferably 5;
a is an integer from 0 to 20, preferably 0 to 10, more preferably 0 to 4, and most preferably 0; and
b is an integer from 0 to 10, preferably 0 to 5, more preferably 0 to 2, and most preferably 0;
with the proviso that the following equation is fulfilled:
n+a+2*b=S*m, wherein S is the value of the oxidation state of M, preferably selected from 1, 2, 3, 4 and 5, more preferably selected from 2, 3, 4 and 5, and most preferably 5.

Preferably, the metal complex is represented by Formula (3), wherein:
M is at each occurrence independently from each other a metal selected from the list consisting of V, Nb and Ta, preferably Nb;
L is at each occurrence independently from each other an organic ligand comprising a carboxylic acid ester group and a carboxylic acid group, which is optionally deprotonated;
A is a bridging ligand selected from μ-L⁻, μ-OH⁻, μ-OR⁻, μ-F⁻ μ-Cl⁻, μ-Br⁻ or μ-I⁻, wherein R is an alkyl group having 1 to 5 carbon atoms; and L⁻ is a deprotonated L;
B is a bridging ligand μ-O²⁻;
m is an integer from 1 to 5;
n is an integer from 5 to 25;
a is an integer from 0 to 10; and
b is an integer from 0 to 5;
with the proviso that the following equation is fulfilled:
n+a+2*b=S*m, wherein S is the value of the oxidation state of
M, selected from 1, 2, 3, 4 and 5.

More preferably, the metal complex is represented by Formula (3), wherein:
M is Nb;
L is at each occurrence independently from each other an organic ligand comprising a carboxylic acid ester group and a carboxylic acid group, which is optionally deprotonated;
A is a bridging ligand selected from μ-L⁻, μ-OH⁻, μ-OR⁻, μ-F⁻ μ-Cl⁻, μ-Br⁻ or μ-I⁻, wherein R is methyl, ethyl, propyl, butyl or pentyl; and L⁻ is a deprotonated L;
B is a bridging ligand μ-O²⁻;
m is 1 or 2;
n is an integer from 5 to 10;
a is an integer from 0 to 4; and
b is an integer from 0 to 2;
with the proviso that the following equation is fulfilled:
n+a+2*b=S*m, wherein S is the value of the oxidation state of M, selected from 1, 2, 3, 4 and 5.

Most preferably, the metal complex is represented by Formula (3a):

MLₙ Formula (3a)
wherein:
M is Nb;
L is at each occurrence independently from each other an organic ligand comprising a carboxylic acid ester group and a carboxylic acid group, which is optionally deprotonated; and
n is 5.

### Formulation

The present invention further relates to a formulation, wherein the formulation comprises:
(i) a metal complex according to the present invention as metal oxide precursor;
(ii) an epoxy resin mixture; and
(iii) a photoinitiator.

Preferred metal complexes, which can be used in the formulation according to the present invention are described above.

In a preferred embodiment of the present invention, the weight ratio of the (i) metal complex in the formulation is in the range from 0.1% to 50% (w/w), preferably 0.5% to 30% (w/w), more preferably 1% to 10% (w/w), and most preferably, 5% (w/w), based on the total mass of the formulation.

In a preferred embodiment of the present invention, the weight ratio of the (ii) epoxy resin mixture in the formulation is in the range from 0.1% to 50% (w/w), preferably 0.5% to 30% (w/w), more preferably 1% to 10% (w/w), most preferably 5% (w/w), based on the total mass of the formulation.

In a particularly preferred embodiment of the present invention, the weight ratios of the (i) metal complex and the (ii) epoxy resin mixture in the formulation are the same and are selected from the range from 1% to 10% (w/w), more preferably they are 5% (w/w) each.

In a preferred embodiment of the present invention, the ratio of the weight ratios (w/w) of the (ii) epoxy resin mixture and the (i) metal complex in the formulation is in the range from 1:1 to 1:2, preferably from 1:1.3 to 1:1.8, more preferably from 1:1.6 to 1:1.7.

In a preferred embodiment of the present invention, the (ii) epoxy resin mixture comprises
(ii-1) one or more fluorene epoxy resins; and
(ii-2) one or more acrylate epoxy resins.

In a more preferred embodiment of the present invention, the (ii) epoxy resin mixture comprises
(ii-1) two or more fluorene epoxy resins; and
(ii-2) one or more acrylate epoxy resins.

In a particularly preferred embodiment of the present invention, the (ii) epoxy resin mixture comprises
(ii-1) two fluorene epoxy resins; and
(ii-2) one acrylate epoxy resin.

In a most preferred embodiment of the present invention, the (ii) epoxy resin mixture consists of
(ii-1) two fluorene epoxy resins; and
(ii-2) one acrylate epoxy resin.

Preferably, the (ii-1) fluorene epoxy resins are represented by the following Formula (4):

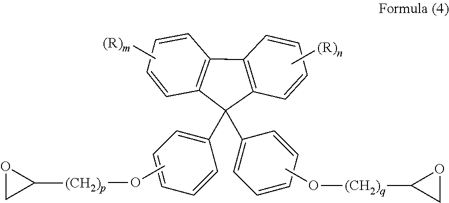

wherein:
R is a halogen, preferably F or Cl, an alkyl group having 1 to 20 carbon atoms, preferably 1 to 10 carbon atoms, more preferably 1 to 5 carbon atoms, or an aryl group having 5 to 20 aromatic ring atoms, preferably 5 to 10 aromatic ring atoms, more preferably 5 or 6 aromatic ring atoms;
m is an integer from 0 to 4, preferably 0 or 1, more preferably 0;
n is an integer from 0 to 4, preferably 0 or 1, more preferably 0;
p is an integer from 0 to 10, preferably 1 to 6, more preferably 1 to 3, most preferably 1; and
q is an integer from 0 to 10, preferably 1 to 6, more preferably 1 to 3, most preferably 1.

More preferably, the (ii-1) fluorene epoxy resins are represented by Formula (4), wherein:
R is F, Cl, an alkyl group having 1 to 10 carbon atoms or an aryl group having 5 to 10 aromatic ring atoms;
m is 0 or 1;
n is 0 or 1;
p is an integer from 1 to 6; and
q is an integer from 1 to 6.

Particularly preferably, the (ii-1) fluorene epoxy resins are represented by Formula (4), wherein:
R is an alkyl group having 1 to 5 carbon atoms or an aryl group having 5 or 6 aromatic ring atoms;
m is 0 or 1;
n is 0 or 1;
p is an integer from 1 to 3; and
q is an integer from 1 to 3.

Most preferably, the (ii-1) fluorene epoxy resins are represented by Formula (4), wherein:
m is 0;
n is 0;
p is 1; and
q is 1.

Preferred (ii-1) fluorene epoxy resins are “OGSOL” (registered trademark) PG 100, EG 200, CG 500, CG 500H, EG 280 and CG 400 (trade names, manufactured by Osaka Gas Chemicals Co., Ltd.).

Preferably, the (ii-2) acrylate epoxy resins are represented by the following Formula (5):

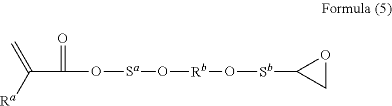

wherein:
R^{a} is H or an alkyl group having 1 to 5 carbon atoms, preferably H or CH₃;
R^{b} is a divalent organic group, preferably selected from an aromatic, aliphatic, or mixed aromatic and aliphatic group, which preferably has 6 to 20 carbon atoms, more preferably —C₆H₄—C(CH₃)₂—C₆H₄—, most preferably -p-C₆H₄—C(CH₃)₂-μ-C₆H₄—;
S^{a} is an alkylene group having 1 to 10 carbon atoms, preferably 1 to 6 carbon atoms, more preferably 1 to 3 carbon atoms, which may be substituted by F or OH; and
S^{b} is an alkylene group having 1 to 10 carbon atoms, preferably 1 to 6 carbon atoms, more preferably 1 to 3 carbon atoms, which may be substituted by F or OH.

More preferably, the (ii-2) acrylate epoxy resins are represented by Formula (5), wherein:
R^{a} is H or an alkyl group having 1 to 5 carbon atoms, preferably H or CH₃;
R^{b} is a divalent organic group selected from an aromatic, aliphatic, or mixed aromatic and aliphatic group, which preferably has 6 to 20 carbon atoms;
S^{a} is an alkylene group having 1 to 6 carbon atoms, which may be substituted by F or OH; and
S^{b} is an alkylene group having 1 to 6 carbon atoms, which may be substituted by F or OH.

Particularly preferably, the (ii-2) acrylate epoxy resins are represented by Formula (5), wherein:
R^{a} is H or CH₃;
R^{b} is a mixed aromatic and aliphatic group, which has 6 to 20 carbon atoms;
S^{a} is an alkylene group having 1 to 3 carbon atoms, which may be substituted by OH; and
S^{b} is an alkylene group having 1 to 3 carbon atoms, which may be substituted by OH.

Most preferably, the (ii-2) acrylate epoxy resins are represented by Formula (5), wherein:
R^{a} is H;
R^{b} is —C₆H₄—C(CH₃)₂—C₆H₄—, preferably -p-C₆H₄—C(CH₃)₂-p-C₆H₄—;
S^{a} is —CH₂—CH(OH)—CH₂—; and
S^{b} is —CH₂—.

A preferred (ii-2) acrylate epoxy resin is CN153.

Preferably, the (iii) photoinitiator is a photoacid generator (PAG), which catalyzes radiation-induced polymerization of the epoxy resin mixture. Preferred photoacid generators (PAGs) are diazonium salts, iodonium salts or sulfonium salts, preferably sulfonium salts, more preferably triaryl sulfonium salts.

Preferred photoacid generators (PAGs) are selected from the list consisting of 4-nitrobenzenediazonium tetrafluoroborate,
bis(4-tert-butylphenyl)iodonium hexafluorophosphate,
bis(4-fluorophenyl)iodonium trifluoromethanesulfonate,
diphenyliodonium hexafluorophosphate,
diphenyliodonium hexafluoroarsenate,
diphenyliodonium trifluoromethanesulfonate,
4-isopropyl-4′-methyldiphenyliodonium tetrakis(pentafluorophenyl)borate,
(4-nitrophenyl)(phenyl)iodonium trifluoromethanesulfonate,
[3-(trifluoromethyl)phenyl](2,4,6-trimethylphenyl)iodonium trifluoromethanesulfonate,
[4-(trifluoromethyl)phenyl](2,4,6-trimethylphenyl)iodonium trifluoromethanesulfonate,
(2-methylphenyl)(2,4,6-trimethylphenyl)iodonium trifluoromethanesulfonate,
(3-methylphenyl)(2,4,6-trimethylphenyl)iodonium trifluoromethanesulfonate,
(4-methylphenyl)(2,4,6-trimethylphenyl)iodonium trifluoromethanesulfonate,
cyclopropyldiphenylsulfonium tetrafluoroborate,
cyclopropyldiphenylsulfonium tetrakis(pentafluorophenyl)borate,
dimethylphenacylsulfonium tetrafluoroborate,
dimethylphenacylsulfonium tetrakis(pentafluorophenyl)borate,
triphenylsulfonium tetrafluoroborate,
triphenylsulfonium bromide,
triphenylsulfonium tetrakis(pentafluorophenyl)borate,
tri-p-tolylsulfonium hexafluorophosphate,
tri-p-tolylsulfonium trifluoromethanesulfonate, and
tri-p-tolylsulfonium tetrakis(pentafluorophenyl)borate.

A particularly preferred (iii) photoacid generator is CPI-310B, i.e. triphenylsulfonium tetrakis(pentafluorophenyl)borate, where one or more of the three phenyl groups are optionally substituted identically or differently, preferably by linear, branched or cyclic alkyl and/or aryl groups.

In a preferred embodiment of the present invention, the formulation further comprises (iv) one or more solvents.

The solvents are selected to improve applicability, wettability, deposition properties, filling properties and/or stability of the formulation. Any solvent can be used for the formulation according to the present invention as long as it dissolves or disperses the components of the formulation. Preferred solvents are selected from water, organic solvents and mixtures thereof. Preferred organic solvents are alcohols, esters, ketones, lactones, diketones, carboxylic acids, amides and mixtures thereof. Particularly preferred organic solvents are ethanol, propanol, 1-butanol, 2-butanol, diacetone alcohol, 1-methoxy-2-propanyl acetate (PGMEA), 1-methoxy-2-propanol (PGME), butyl acetate, amyl acetate, cyclohexyl acetate, 3-methoxybutyl acetate, methyl ethyl ketone, methyl isobutyl ketone, methyl amyl ketone, cyclohexanone, cyclopentanone, ethyl-3-ethoxy propanoate, methyl-3-ethoxy propanoate, methyl-3-methoxy propanoate, methyl acetoacetate, ethyl acetoacetate, methyl pivalate, ethyl pivalate, propylene glycol monomethyl ether, propylene glycol monoethyl ether, propylene glycol monomethyl ether propanoate, propylene glycol monoethyl ether propanoate, ethylene glycol monomethyl ether, ethylene glycol monoethyl ether, diethylene glycol monomethyl ether, diethylene glycol monoethyl ether, 3-methyl-3-methoxybutanol, N-methylpyrrolidone, dimethyl sulfoxide, gamma-butyrolactone, gamma valerolactone, propylene glycol methyl ether acetate, propylene glycol ethyl ether acetate, propylene glycol propyl ether acetate, methyl lactate, ethyl lactate, propyl lactate, tetramethylene sulfone, propylene glycol dimethyl ether, dipropylene glycol dimethyl ether, ethylene glycol dimethyl ether or diethylene glycol dimethyl ether, N-ethylpyrrolidone, 2-pyrrolidone, 2,2,4-trimethyl-1,3-pentandiol monoisobutyrate, terpineol, 3-phenoxytoluene, 4-phenoxytoluene and anisole. These solvents may be used singly or in a mixture of two or more.

In a preferred embodiment of the present invention, the formulation comprises one or more further metal complexes, which may act as further metal oxide precursors. In such case, a mixed metal oxide optical layer may be formed comprising a metal oxide obtained from the metal complex according to the present invention and a further metal oxide obtained from the further metal complexes.

Preferred further metal complexes comprise one or more trivalent or tetravalent metals, preferably selected from the list consisting of Sc, Y, La, Ti, Zr, Hf and Sn, more preferably one or more tetravalent metals selected from the list consisting of Ti, Zr, Hf and Sn.

In a preferred embodiment of the present invention, the formulation comprises one, two, three, four or more further metal complexes in addition to the metal complex, where preferably each of the further metal complexes contains ligands selected from inorganic ligands or organic ligands. Preferred inorganic ligands are halogenides, phosphoric acid, sulfonic acid, nitric acid and water, which are optionally deprotonated. Preferred organic ligands are alcohols, carboxylic acids, cyanates, isocyanates, 1,3-diketones, beta-keto acids, beta-keto esters, organylphosphonic acids, organylsulfonic acids, oximes, hydroxamic acids, dihydroxy benzenes, hydroxybenzoic acids, dihydroxy benzoic acids, gallic acid, dihydroxynaphthalenes, anthracene diols, hydroxy-anthrones, anthracene triols, dithranols, halogenated hydrocarbons, aromatics, heteroaromatics, esters, catechols, coumarins and their derivatives, which are optionally deprotonated.

The presence of such further metal complexes allows to adjust certain properties of the metal oxide optical layer prepared therefrom such as e.g. material hardness, shrinkage, refractive index, transparency, absorbance, and haze suppression.

Preferably, the weight ratio (w/w) between the metal complex according to the present invention and the one or more further metal complexes in the formulation is in the range from 1:100 to 100:1, preferably from 1:10 to 10:1, and more preferably from 1:5 to 5:1.

It is preferred that the total weight ratio of the metal complex according to the present invention and the further metal complexes contained in the formulation is in the range from 0.1% to 50% (w/w), preferably 0.5% to 30% (w/w), more preferably 1% to 10% (w/w), most preferably 5% (w/w), based on the total mass of the formulation.

In a preferred embodiment of the present invention, the formulation is an ink formulation being suitable for inkjet printing. Typical requirements for ink formulations are surface tensions in the range from 20 mN/m to 30 mN/m and viscosities in the range from 5 mPa·s to 10 mPa·s.

### Method for Preparing Metal Oxide Optical Layer

The present invention further relates to a method for preparing a metal oxide optical layer, wherein the method comprises the following steps:
(a) providing a formulation according to the present invention;
(b) applying the formulation to a surface of a substrate; and
(c) converting the formulation on the surface of the substrate to a metal oxide optical layer.

Preferably, the formulation provided in step (a) is a solution or dispersion comprising one or more solvents. Preferred solvents for the formulation provided in step (a) are the same as described above for the formulation according to the present invention.

In a preferred embodiment of the present invention, the formulation provided in step (a) is an ink formulation being suitable for inkjet printing. Typical requirements for ink formulations are surface tensions in the range from 20 mN/m to 30 mN/m and viscosities in the range from 5 mPa·s to 10 mPa·s.

Preferably, the formulation is applied in step (b) to a surface of a substrate by a deposition method. A preferred deposition method is drop casting, coating, or printing. A more preferred coating method is spin coating, spray coating, slit coating, or slot-die coating. A more preferred printing method is flexo printing, gravure printing, inkjet printing, EHD printing, offset printing, or screen printing. Most preferred are spray coating and inkjet printing.

Depending on the specific problem to be solved, the formulation needs to be deposited either as a homogeneous, dense and thin layer covering the entire surface of the substrate by a coating method or the formulation needs to be deposited locally in a structured manner, thus requiring for a printing method. Both, coating and printing methods require formulations to be formulated in an adequate manner to comply with the physico-chemical needs of the respective coating and printing method as well as to comply with certain needs regarding the surface of the substrate to be coated or printed.

In a preferred embodiment of the method for preparing a metal oxide optical layer according to the present invention, the surface of the substrate is pre-treated by a surface cleaning process. Preferred surface cleaning processes are silicon wafer cleaning processes such as described in W. Kern, The Evolution of Silicon Wafer Cleaning Technology, J. Electrochem. Soc., Vol. 137, 6, 1990, 1887-1892 and in New Process Technologies for Microelectronics, RCA Review 1970, 31, 2, 185-454. Such silicon wafer cleaning processes include wet cleaning process involving cleaning solvents (e.g. isopropanol (IPA)); wet etching processes involving hydrogen peroxide solutions (e.g. piranha solution, SC1, and SC2), choline solutions, or HF solutions; dry etching processes involving chemical vapor etching, UV/ozone treatments or glow discharge techniques (e.g. 02 plasma etching); and mechanical processes involving brush scrubbing, fluid jet or ultrasonic techniques (sonification). The surface of the substrate can also be pre-treated by silanization or an atomic layer deposition (ALD) process. The pre-treatment of the surface of the substrate serves to modify the hydrophobicity/hydrophilicity of the surface. This can improve the adhesion and filling characteristics of the metal oxide optical layer on the surface of the substrate.

In a more preferred embodiment, a wet cleaning process involving cleaning solvents (e.g. isopropanol (IPA)) is combined with one or more of a wet etching process involving hydrogen peroxide solutions (e.g. piranha solution, SC1, and SC2), choline solutions, or HF solutions; dry etching process involving chemical vapor etching, UV/ozone treatments or glow discharge techniques (e.g. O₂ plasma etching); and mechanical process involving brush scrubbing, fluid jet or ultrasonic techniques (sonification).

In a most preferred embodiment, a wet cleaning process involving cleaning solvents (e.g. isopropanol (IPA)) is combined with a mechanical process involving brush scrubbing, fluid jet or ultrasonic techniques (sonification) and with a wet etching process involving hydrogen peroxide solutions (e.g. piranha solution, SC1, and SC2), choline solutions, or HF solutions;

In a preferred embodiment of the present invention, step (b) of the method for preparing a metal oxide optical layer is carried out once.

In a preferred embodiment of the present invention, step (b) of the method for preparing a metal oxide optical layer is carried out several times in succession, preferably 2 to 20 times, more preferably 2 to 10 times, most preferably 2, 3, 4 or 5 times.

In a preferred embodiment of the method for preparing a metal oxide optical layer according to the present invention, the formulation is converted in step I on the surface of the substrate to a metal oxide optical layer by exposure to irradiation, preferably in combination with thermal treatment.

Preferred exposure to irradiation includes exposure to infrared (IR) light, visible (Vis) light and/or ultraviolet (UV) light. IR light has a wavelength of >800 nm. Vis light has a wavelength from 400 to 800 nm. UV light has a wavelength of <400 nm and may include EUV (extreme UV). Exposure to Irradiation is not limited to any specific irradiation methods or times. Depending on the type of substrate and formulation, a person skilled in the art is able to determine suitable irradiation methods and times.

Preferred thermal treatment includes exposure to elevated temperatures as high as 400° C., preferably up to 350° C., preferably up to 300° C., more preferably up to 250° C., particularly preferably up to 200° C. and most preferably up to 150° C. Thermal treatment is not limited to any specific thermal treatment methods or times. Depending on the type of substrate and formulation, a person skilled in the art is able to determine suitable thermal treatment methods and times.

In a more preferred embodiment of the method for preparing a metal oxide optical layer according to the present invention, the formulation is converted in step I on the surface of the substrate to a metal oxide optical layer by (c-1) pre-baking (soft baking) at a temperature from 40 to 150° C., preferably from 50 to 120° C., more preferably from 60 to 100° C.; by (c-2) exposure to irradiation, preferably UV irradiation; and by (c-3) baking (hard baking, sintering or annealing) at a temperature from 100 to 400° C., preferably from 150 to 350° C., more preferably from 200 to 250° C.

Pre-baking (soft baking) serves the purpose to remove volatile and low boiling components such as e.g. volatile and low boiling solvents or additives from the drop casted, coated or printed films. Pre-baking is preferably carried out for a period of 1 to 10 minutes. After pre-baking, layers of substrate adhering films of the formulation are obtained. The films may still comprise residual solvents or additives.

In an alternative more preferred embodiment of the method for preparing a metal oxide optical layer according to the present invention, (c-1) pre-baking can be omitted so that the formulation is converted in step I on the surface of the substrate to a metal oxide optical layer directly by (c-2) exposure to irradiation, preferably UV irradiation; and by (c-3) baking (hard baking, sintering or annealing) at a temperature from 100 to 400° C., preferably from 150 to 350° C., more preferably from 200 to 250° C.

Exposure to irradiation (curing) serves the purpose to crosslink the epoxy resin mixture contained in the formulation, thereby creating a polymer matrix. Said polymer matrix acts as a skeleton that prevents the formation of voids due to evaporation of the ligands of the metal complex and subsequent shrinkage of the material. Preferred exposure to irradiation involves exposure to UV light, preferably to UV light having a wavelength of 365 nm, for 1 to 60 minutes, preferably 5 to 30 minutes, and more preferably 10 to 20 minutes.

Baking (hard baking, sintering or annealing) serves the purpose to convert the formulation on the surface of the substrate into a metal oxide layer. Moreover, the final properties of the metal oxide layer may be adjusted by the baking treatment. Baking is preferably carried out for a period of 1 to 60 minutes, preferably 1 to 30 minutes, more preferably 5 to 20 minutes, and most preferably 10 minutes, to achieve a refractive index (RI) of >2.0.

Pre-baking, exposure to irradiation and baking may be carried out under ambient atmosphere or atmospheres with increased oxygen content in order to decompose unwanted organic components, which can lead to a lower activation energy when the metal oxide layers are formed.

In a preferred embodiment of the method for preparing a metal oxide optical layer according to the present invention, the substrate is a patterned substrate comprising topographical features on the surface thereof and the metal oxide optical layer forms a coating layer covering the surface of the substrate and filling said topographical features. As a result, the topographical features are filled and levelled by said metal oxide optical layer.

Preferred topographical features include, for example, gaps, grooves, trenches and vias. Topographical features may be distributed uniformly or non-uniformly over the surface of the substrate. Preferably, they are arranged as an array or grating on the surface of the substrate. It is preferred that the topographical features have different lengths, widths, diameters as well as different aspect ratios. It is preferred that said topographical features have an aspect ratio of 1:20 to 20:1, more preferably 1:10 to 10:1. The aspect ratio is defined as width of structure to its height (or depth). From the viewpoint of dimension, the depth of the topographical features is preferably in the range from 10 nm to 10 μm, more preferably 50 nm to 5 μm, and most preferably 100 nm to 1 μm.

It is also preferred that the topographical features are inclined at a certain angle, such as an angle from 10 to 80°, preferably from 20 to 60°, more preferably from 30 to 50°, most preferably about 40°. Such inclined topographical features are also referred to as slanted or blazed topographical features.

It may be also necessary to fill topographical features locally with metal oxide optical layer, either completely or to a certain level, but not to cover adjacent surfaces of the substrate, where no topographical features to be filled are available.

Hence, it is preferred that the method for preparing a metal oxide optical layer according to the present invention further comprises the following step (d):
(d) removing a portion of said metal oxide optical layer covering the top of the topographical features, thereby obtaining filled topographical features, wherein an overburden of the metal oxide optical layer on top of said topographical features is reduced, preferably to an overburden of between 0 to 100 nm, more preferably between 0 to 50, and most preferably between 0 to 20 nm.

Step (d) takes place after steps (a) to (c) of the method according to the present invention. Preferably, removing a portion of said metal oxide optical layer covering a top of the topography in step (d) is performed by using a surface cleaning process as described above. Preferred surface cleaning processes are silicon wafer cleaning processes such as described in W. Kern, The Evolution of Silicon Wafer Cleaning Technology, J. Electrochem. Soc., Vol. 137, 6, 1990, 1887-1892 and in New Process Technologies for Microelectronics, RCA Review 1970, 31, 2, 185-454. Such silicon wafer cleaning processes include wet-etching processes involving hydrogen peroxide solutions (e.g. piranha solution, SC1, and SC2), choline solutions, or HF solutions; dry-etching processes involving chemical vapor etching, UV/ozone treatments or glow discharge techniques (e.g. O₂ plasma etching); and mechanical processes involving brush scrubbing, fluid jet or ultrasonic techniques.

The substrate is preferably a substrate of an optical device. Preferred substrates are made of inorganic or organic base materials, preferably inorganic base materials. Preferred inorganic base materials contain materials selected from the list consisting of ceramics, glass, fused silica, sapphire, silicon, silicon nitride, quartz, and transparent polymers or resins. The geometry of the substrate is not specifically limited, however, preferred are sheets or wafers.

In step (b) of the method for preparing a metal oxide optical layer, the formulation is applied on a surface of a substrate, wherein said surface may be either a surface of a base material of the substrate or a surface of a layer of a material being different from the base material of the substrate, wherein such layer has been formed prior to applying said formulation.

In this way, sequences of different layers (layer stacks) can be formed on top of one another. Such layer stacks may be also structured, wherein such structures typically have dimensions in the nanometer scale, at least with respect to diameter, width and/or aspect ratio.

### Optical Device

Finally, the present invention relates to an optical device comprising a metal oxide optical layer, which is obtainable or obtained by the method for preparing a metal oxide optical layer according to the present invention as described above. It is preferred that the optical device is an augmented reality (AR) and/or virtual reality (VR) device.

The present invention is further illustrated by the examples following hereinafter which shall in no way be construed as limiting. The skilled person will acknowledge that various modifications, additions and alternations may be made to the invention without departing from the spirit and scope of the invention as defined in the appended claims.

### EXAMPLES

### Analytics and Measurement Methods

Ellipsometry was used to determine layer thickness, refractive index (n) and absorption index (k) of a metal oxide layer. Measurements were performed using an ellipsometer M2000 from J. A. Woolam and three different angles of incidence (65°, 70° and 75°). The measurement data was analyzed with software CompleteEase from J. A. Woolam, assuming either full or almost nearly complete transparent behavior above a wavelength of 600 nm and applying B-spline fitting for obtaining refractive indices (n) as well as absorption indices (k). The optical constants were averaged from three to four measured samples each of them providing a different layer thickness either after soft bake or after hard bake or after combined soft and subsequent hard bake.

Optical spectra of any sheets and substrates being either coated or uncoated by metal oxide layers described in the present invention were recorded using UV/Vis/NIR-spectrophotometer Cary 7000 from Agilent with UMA-setup. Measurements were carried out using dual beam mode, a scan speed of 600 nm/min and a spectral band width of 4 nm, non-polarized light and applying a spectral window from 350 nm to 700 nm. Transmission measurements were carried out with an angle of incidence of 6° versus surface normal of the sample. The detector was aligned 1800 to light incidence. Reflection measurements were carried out with an angle of incidence of 6° versus surface normal of the sample, the detector angle amounted to 12° versus incidence of light. The absorption of the samples was calculated using Equation 1, where A stands for the absorption of the coated sample, R stands for the reflection and T for the transmission of the sample.

$$A = 1 - ( R + T ) \qquad Equation 1$$

Thermogravimetric analysis was run on a TGA Q 50 from TA Instruments. In the usual measurement mode, the sample was heated up to 950° C. in air atmosphere applying a heating ramp of 20 K/min.

Results upon elementary analysis were received as service from an analytical service provider where measurements were conducted according to DIN 51732:2014-07.

NMR-measurements, ¹H-NMR, were measured using 500 MHz spectrometer from Bruker Biospin GmbH.

SEM images were recorded using either a Mira 3 LMU from Tescan or Sigma 300VP from Carl Zeiss or Supra 35 from Carl Zeiss, too.

Substrate coating, usually wafers, was done using a spin coater (LabSpin 150i) from Seuss. The spin coating process using planar substrates was as follows: deposition of 0.5 ml of the coating onto static quartz wafers followed by a spinning interval of 30 seconds at a given spin speed where the acceleration to reach the final spin speed was set to 500 rpm/s². Different layer and coating thicknesses were achieved using either different spin speeds or different coating formulations having different concentrations of the metal oxide precursor or mixtures of different metal oxide precursors. After spin coating, the coated substrates either underwent pre-baking at 100° C. for 2 minutes for driving out solvent residues, subsequently followed by baking at elevated temperatures or the layers deposited on the wafers became directly baked at elevated temperature for a dedicated time. Usually, however not limited hereto, the coated layers were baked at 150° C., 200° C., 250° C. and 300° C. for typically 5 minutes to 10 minutes. Pre-baking as well as layer baking were performed using high temperature hotplates from Harry Gestigkeit allowing for reaching temperatures of up to 600° C. Aforementioned conditions and parameters apply to all following experimental examples unless other conditions are explicitly mentioned elsewhere.

Usually, quartz and/or silicon wafers, both 2″ in diameter, were used throughout all coating experiments where flat and non-structured carriers for metal oxides were required (e. g. spectroscopic and ellipsometry measurements).

Structured substrates, usually silicon wafers, were used as square-shaped dies with edge length of 1.5 cm to 2 cm. The wafer dies were cut and cleaved from a parent wafer, typically having a diameter of 8″. The structures were created and arranged in a layer stack composed of SiO₂/SiNₓ being deposited onto the wafer surface. Dimensions of the structures (e. g. cross-section width and length of trenches) referred to the architecture of Sematech mask 854. Usually, however not limited hereto, the cross-sectional cleaves perpendicular to trench arrays providing a width of 40 nm to 50 nm were used as trench structures of primary interest to investigate their filling behavior by the wet-chemically coated metal oxide precursors and/or metal oxides received upon thermal conversion of the said metal oxide precursors. Besides to aforementioned, cross-sections of arrays of trenches having widths of 100 nm and 150 nm where used to investigate trench filling by metal oxides, too.

Structured wafer dies were, unless otherwise mentioned, coated by spin coating. For that purpose, the coating formulation, typically a volume between 0.15 ml to 0.5 ml per die (having a diameter of 0.5″ to 2″), was pipetted and casted onto wafer's surface. The formulation was allowed to spread and settle on the surface for one minute followed by a step of distributing and spreading of the formulation over the entire surface of the wafer die at 500 rpm for 30 seconds, followed by a final spin-off step at 2,000 rpm for further 60 seconds. The acceleration of the spin speed was set to 500 rpm/s². The soft bake and hard conditions of structured wafer dies was chosen similar or identical to those already mentioned for flat substrates.

All chemicals for synthesis described were purchased from Sigma Aldrich and used without further purification, unless differently mentioned elsewhere.

### Preparation of Nb Citraconate

A 3-necked glass flask (100 ml) was continuously flushed with N2 and 17.6116 g citraconic acid anhydride as well as 8.160 g 1-methoxy-2-propanol acetate (PGME) were added into the flask (first solution). The mixture was allowed to stir for 15 min under a constant stream of N2. In parallel, a mixture of 28.770 g 1-methoxy-2-propanylacetate (PGMEA) and 10 g of niobium ethoxide (second solution) was prepared under inert atmosphere and added over a period of 1 h to the first solution flask using a dripping funnel. After completion of the addition of the second solution to the first solution, the reaction mixture was heated to 50° C. under continuous stirring over a period of 4 h. Afterwards, the mixture was allowed to cool down and filled into an appropriate inertized brown glass bottle. A small volume of the material was withdrawn and the solvent was removed and the remaining residue was dried until reaching a final pressure of 0.01 mbar in the sample vial. The remaining crude product was subjected to 1H-NMR analysis: 6.78 ppm (s, 1H), 5.81 ppm (m, 0.56H), 4.16 ppm (q, 0.56H), 4.10 ppm (q, 0.82H), 3.29 ppm (m, 3.04H), 2.12 ppm (s, 3.36H), 1.98 ppm (m, 2.06H), 1.18 ppm (m, 5.64H).

### Example 1

### Formulation 1:

Epoxy Mixture A (see Table 1 below) was mixed with 10% (w/w) Nb citraconate as prepared above in PGME in a 1:1 weight ratio, resulting in a final concentration of Epoxy Mixture A and Nb citraconate of 5% (w/w) each, i.e. 5/5% (w/w) Nb citraconate/Epoxy Mixture A.

### Epoxy Mixture A:

TABLE 1
Epoxy Mixture A.

| component | weight (g) | % (w/w) |
|---|---|---|
| PGME | 4.4988 | 89.9 |
| OGSOL CG 500 | 0.2496 | 5.0 |
| OGSOL EG 200 | 0.2021 | 4.0 |
| CN153 | 0.0505 | 1.0 |
| CPI-310B | 0.005 | 0.1 |

### Deposition:

100 μL of Formulation 1 was deposited on the substrate (18×18 mm) with surface features by deposition of 100 μL drop and subsequent spin coating at 2,000 rpm for 25 sec. The coated substrate was then prebaked at 100° C. for 1 min. After that the substrate was irradiated by 365 UV lamp for 20 min and then baked at 300° C. for 10 min.

The formulation deposited via the process described above fills the surface features.

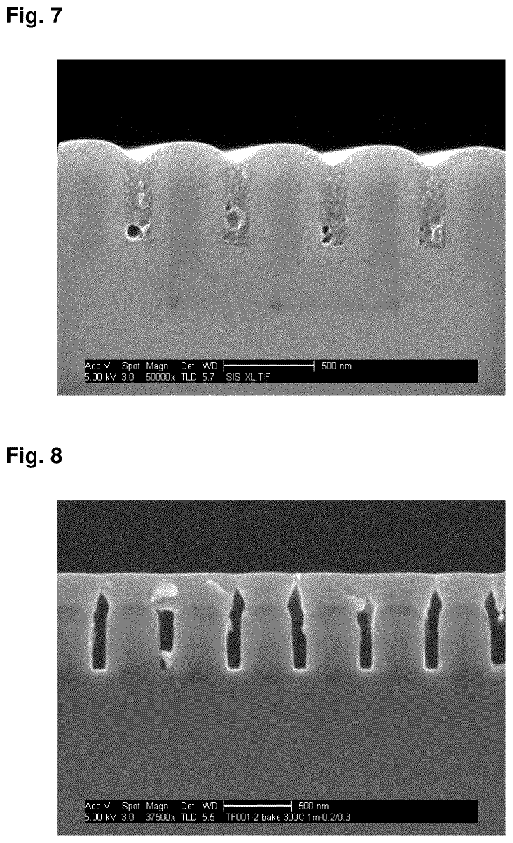

FIG. 7 shows surface feature filling of the spin coated 5/5% (w/w) Nb citraconate/Epoxy Mixture A after prebake at 100° C. for 1 min followed by UV-curing by 365 nm light for 20 min and bake at 350° C. for 10 min.

FIG. 8 shows surface feature filling of the spin coated Nb citraconate 5% (w/w) solution in PGME, annealed at 300° C. for 10 min.

Optical properties (ellipsometry measurements) of the formed films prepared with UV-curing after bake at different temperatures are shown in Table 2 below.

TABLE 2
Optical properties (ellipsometry measurements) of the formed films
prepared with UV-curing after bake at different temperatures.

| Baking temperature | Thickness | n @ 520.0 | k @ 460.0 |
|---|---|---|---|
| and time | (nm) | nm | nm |
| 350° C./10 min | 32 | 2.07 | 0.019 |

### Example 2

### Formulations:

Different weight ratios of Nb citraconate (1:1 in PGME) and Epoxy Mixture A (see Table 1 above) were prepared, spin coated, irradiated by 365 UV lamp for 20 min and then baked at 150° C., 200° C. or 250° C. for 10 mins. The coated films were then tested for gap fill (on structured substrates), RI and absorption (on quartz substrates).

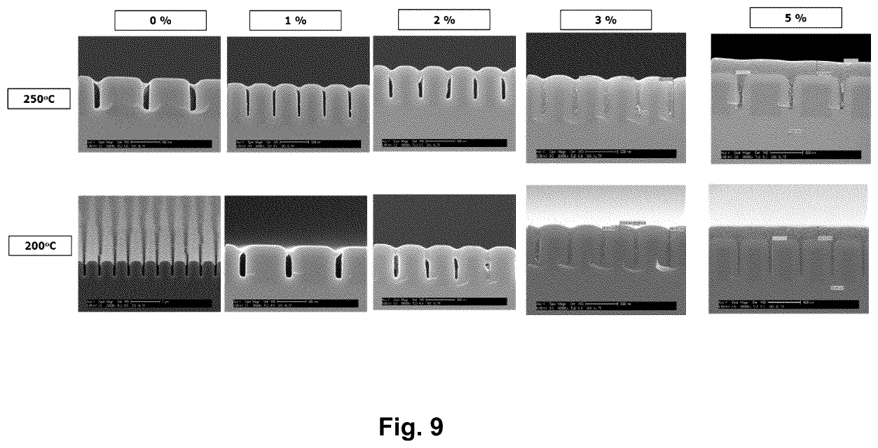

FIG. 9 shows surface feature filling behavior for Nb citraconate+Epoxy Mixture A at various mixing ratios and baking temperatures. The Nb citraconate concentration stays constant at 5% (w/w) and the Epoxy Mixture A content is varied from 1% (w/w) to 5% (w/w) as indicated.

With a ratio of 3% (w/w):5% (w/w) between Epoxy Mixture A: Nb citraconate and a higher content of Epoxy Mixture A, homogeneous gap fill is achieved after baking at 200° C. and 250° C.

From Table 3 it can be seen that for those experiments and material weight ratios where good fill is achieved (3:5 to 5:5), the refractive index n of these films can be tuned between 1.659 and 1.758 by applying different temperatures between 150° C. and 250° C.

Table 4 shows the absorption index k at 460 nm wavelength. Low absorption is achieved for samples baked at 150° C.

TABLE 3
Refractive index n at 520 nm of films of 5% (w/w)
Nb citraconate with varying content of Epoxy Mixture
A (as indicated) after baking at 150° C.-250° C.

| | | Baking temperature/n | | |
|---|---|---|---|---|
| | | 150° C. | 200° C. | 250° C. |
| Epoxy Mixture | 0 | 1.637 | 1.643 | 1.810 |
| A % (w/w) | 1 | 1.673 | 1.677 | — |
| | 2 | 1.668 | 1.681 | 1.709 |
| | 3 | 1.666 | 1.700 | 1.704 |
| | 4 | 1.659 | 1.677 | 1.763 |
| | 5 | 1.664 | 1.672 | 1.758 |

TABLE 4
Absorption index k at 460 nm of films of 5% (w/w)
Nb citraconate with varying content of Epoxy Mixture
A (as indicated) after baking at 150° C.-250° C.

| Baking | Epoxy Mixture A % (w/w)/k | | | | | |
|---|---|---|---|---|---|---|
| temperature | 0 | 1 | 2 | 3 | 4 | 5 |
| 150° C. | 0.055 | 0.011 | 0.001 | 0.0155 | 0.0545 | 0.153 |
| 200° C. | 0.0555 | 0.154 | 0.055 | 0.4295 | 0.33 | 0.3445 |
| 250° C. | 0.038 | 0.2425 | 0.2655 | 0.877 | 0.706 | 0.173 |

### Example 3

The following solutions ((5% (w/w) each): Ti isopropoxide, Ti butoxide, Nb ethoxide and Nb citraconate were each spin coated on silicon wafers and baked at 350° C. for 10 mins. In addition, new formulations based on mixtures of Ti isopropoxide/Epoxy Mixture A (5/5% (w/w)), Ti butoxide/Epoxy Mixture A (5/5% (w/w)), Nb ethoxide/Epoxy Mixture A (5/5% (w/w)) and Nb citraconate/Epoxy Mixture A (5/5% (w/w)) were coated on silicon wafers under the same conditions as mentioned above. The refractive index and absorption index of the coated films were measured.

### Measurement of Refractive Index n and Absorption Index k

Table 5 shows that a refractive index n greater 2.00 can be achieved with various metal-organic metal oxide precursors. However, all of these materials, except Nb citraconate, give a significant increase in the absorption index k with the addition of Epoxy Mixture A. Only for Nb citraconate, k decreases when mixed with Epoxy Mixture A.

TABLE 5
Refractive index (n) and absorption index (k) of various
metal oxide precursors with and without Epoxy Mixture A
in a 5/5% (w/w) ratio each after 350° C. baking for 10 mins.

| Formulation | n | k |
|---|---|---|
| Ti isopropoxide (5% (w/w)) | 2.296 | 0.0155 |
| Ti isopropoxide/Epoxy Mixture A (5/5% | 2.02 | 0.0346 |
| (w/w)) | | |
| Ti butoxide (5% (w/w)) | 2.193 | 0.00717 |
| Ti butoxide/Epoxy Mixture A (5/5% (w/w)) | 2.102 | 0.2109 |
| Nb ethoxide (5% (w/w)) | 2.157 | 0.0683 |
| Nb ethoxide/Epoxy Mixture A (5/5% (w/w)) | 2.044 | 0.0929 |
| Nb citraconate (5% (w/w)) | 2.065 | 0.05043 |
| Nb citraconate/Epoxy Mixture A (5/5% | 2.07 | 0.019 |
| (w/w)) | | |

Since k is proportional to the optical loss of a film, only formulations comprising Nb citraconate achieve the preferable and surprising effects for use in optical coatings, namely lowering absorption (optical loss) when mixed with an epoxy based resin matrix to improve filling performance.

### LIST OF REFERENCE SIGNS

1 Material 02 with RI 02
2 Material 01 with RI 01
3 Substrate (e.g. glass)
4 Diffraction of incident light represented by broad arrow
5 Total internal reflection of light (TIR)
6 Waveguide
7 Structured layer stack with gaps (trenches)
8 Substrate (e.g. glass or silicon)
9 Overburden of material (e.g. high refractive index material or high etch resistant material)
10 Material (e.g. high refractive index material or high etch resistant material) providing gap fill
11 Voids
12 Formulation (e.g. ink) of high refractive index material (e.g. metal oxide precursor)
13 High refractive index material (e.g. metal oxide) providing gap fill with optional concave geometry
14 Overburden layer (optional)
15 Energy

1. A metal complex comprising:
at least one metal M selected from the list consisting of V, Nb and Ta, preferably Nb; and
at least one ligand L, wherein L is an organic ligand comprising an ester group and a carboxylic acid group, which is optionally deprotonated.

2. The metal complex according to claim 1, wherein L is an organic ligand further comprising a carbon-carbon double bond.

3. The metal complex according to claim 1, wherein L coordinates via the carboxylic acid group to the one or more metals M.

4. The metal complex according to claim 1, wherein L is a compound represented by Formula (1), which is optionally deprotonated at the carboxylic acid group: 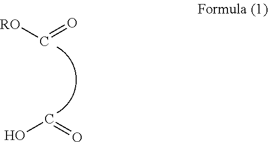
wherein:
the curved line represents a divalent hydrocarbon group having 2 to 20 carbon atoms and one or more carbon-carbon double bonds; and
R is an alkyl group having 1 to 20 carbon atoms or an aryl group having 5 to 20 aromatic ring atoms, which may be substituted or unsubstituted.

5. The metal complex according to claim 1, wherein L is a compound represented by Formula (2), which is optionally deprotonated at the carboxylic acid group: 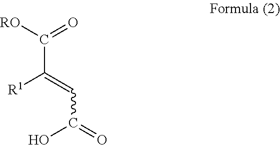
wherein:
R is an alkyl group having 1 to 20 carbon atoms or an aryl group having 5 to 20 aromatic ring atoms, which may be substituted or unsubstituted; and
R¹ is H, an alkyl group having 1 to 10 carbon atoms or an aryl group having 5 to 10 aromatic ring atoms, which may be substituted or unsubstituted.

6. The metal complex according to claim 1, wherein the metal complex is represented by the following Formula (3):
MₘLₙAₐB_{b} Formula (3)
wherein:
M is at each occurrence independently from each other a metal selected from the list consisting of V, Nb and Ta, preferably Nb;
L is at each occurrence independently from each other an organic ligand comprising an ester group and a carboxylic acid group, which is optionally deprotonated;
A is a bridging ligand selected from μ-L⁻, μ-OH⁻, μ-OR⁻, μ-F⁻ μ-Cl⁻, μ-Br⁻ or μ-I⁻, wherein R is an alkyl group having 1 to 10 carbon atoms; and L⁻ is a deprotonated L;
B is a bridging ligand μ-O²⁻;
m is an integer from 1 to 10;
n is an integer from 5 to 50;
a is an integer from 0 to 20; and
b is an integer from 0 to 10;
with the proviso that the following equation is fulfilled:
n+a+2*b=S*m, wherein S is the value of the oxidation state of M and is selected from 1, 2, 3, 4 and 5.

7. A formulation comprising
(i) a metal complex according to claim 1 as metal oxide precursor;
(ii) an epoxy resin mixture; and
(iii) a photoinitiator.

8. The formulation according to claim 7, wherein the weight ratio of the (i) metal complex in the formulation is in the range from 0.1% to 50% (w/w), based on the total mass of the formulation.

9. The formulation according to claim 7, wherein the (ii) epoxy resin mixture comprises
(ii-1) one or more fluorene epoxy resins; and
(ii-2) one or more acrylate epoxy resins.

10. The formulation according to claim 9, wherein the (ii-1) one or more fluorene epoxy resins are represented by the following Formula (4): 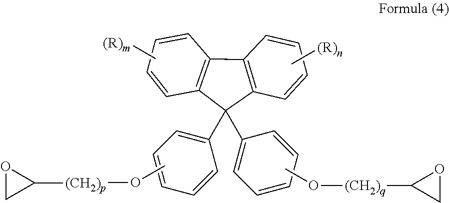
wherein:
R is a halogen, an alkyl group having 1 to 20 carbon atoms or an aryl group having 5 to 20 aromatic ring atoms;
m is an integer from 0 to 4;
n is an integer from 0 to 4;
p is an integer from 0 to 10; and
q is an integer from 0 to 10.

11. The formulation according to claim 9, wherein the (ii-2) one or more acrylate epoxy resins are represented by the following Formula (5): 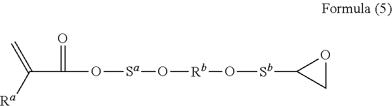
wherein:
R^{a} is H or an alkyl group having 1 to 5 carbon atoms;
R^{b} is a divalent organic group;
S^{a} is an alkylene group having 1 to 10 carbon atoms, which may be substituted by F or OH; and
S^{b} is an alkylene group having 1 to 10 carbon atoms, which may be substituted by F or OH.

12. The formulation according to claim 7, further comprising
(iv) one or more solvents.

13. A method for preparing a metal oxide optical layer comprising the following steps:
(a) providing a formulation according to claim 7;
(b) applying the formulation to a surface of a substrate; and
(c) converting the formulation on the surface of the substrate to a metal oxide optical layer.

14. The method according to claim 13, wherein in step (b) the formulation is applied to a surface of a substrate by a deposition method.

15. The method according to claim 13, wherein in step (c) the formulation is converted on the surface of the substrate to a metal oxide optical layer by exposure to irradiation.

16. The method according to claim 13, wherein in step (c) the formulation is converted on the surface of the substrate to a metal oxide optical layer by (c-1) pre-baking at a temperature from 40 to 150° C.; by (c-2) exposure to irradiation; and (c-3) baking at a temperature from 100 to 400° C.

17. The method according to claim 13, wherein the substrate is a patterned substrate comprising topographical features on the surface thereof.

18. An optical device comprising a metal oxide optical layer, which is obtainable by the method according to claim 13.

19. The optical device according to claim 18, wherein the optical device is an augmented reality (AR) and/or virtual reality (VR) device.

20. The metal complex according to claim 6, wherein M is 5.
